(19) United States
(12) Patent Application Publication
(10) Pub. No.: US 2018/0353742 A1
(43) Pub. Date: Dec. 13, 2018

(54) IMPLANTABLE MEDICAL PORT

(71) Applicant: Charles Rafic, WEHBE, Lakewood, OH (US)

(72) Inventor: Charles Rafic WEHBE, Lakewood, OH (US)

(21) Appl. No.: 15/778,682

(22) PCT Filed: Nov. 18, 2016
(86) PCT No.: PCT/US16/62883

§ 371 Date: May 24, 2018

Related U.S. Application Data
(60) Provisional application No. 62/259,616, filed on Nov. 24, 2015.
(60) Provisional application No. 62/322,527, filed on Apr. 14, 2016.

(51) Int. Cl. A61M 39/02

(52) U.S. Cl. CPC A61M 39/0208; A61M 2039/0226; A61M 2039/022; A61M 2039/0217

(57) ABSTRACT

Disclosed is an implantable medical port and methods of using the implantable medical port to deliver and/or to withdraw a desired agent, such as medicines, treatments, or any other fluid into and/or from a body of a subject. The implantable medical port includes two or more needle-penetrable septum portions to provide an increased needle penetration area. Also provided is an implantable medical port having a fluid cavity the contains a substantially needle-impenetrable member that prevents a needle from inadvertently puncturing or otherwise damaging a septum portion other than the septum portion that was initially punctured to gain access to the fluid cavity.

### CROSS REFERENCE TO RELATED APPLICATIONS

This application claims the benefit of the filing date under 35 U.S.C. § 119(e) of U.S. Provisional Application For Patent Ser. No. 62/259,616 filed on Nov. 24, 2015 and U.S. Provisional Application For Patent Ser. No. 62/322,527 filed Apr. 14, 2016, both of which are incorporated herein by reference.

This disclosure relates to an implantable medical port, a medical device, and methods of using the implantable medical port to deliver and/or to withdraw a desired agent, such as medicines, treatments, or any other fluid into and/or from a body of a subject.

Implantable medical ports, commonly referred to as “medical ports”, “med ports” or “ports”, are utilized to deliver and/or withdraw fluids from a patient. Such ports typically include a cup-shaped needle-impenetrable housing having a needle-penetrable, self-sealing septum mounted over the open end of the cup-shaped housing to define a fluid cavity, otherwise known as a “reservoir compartment”. The needle-penetrable septum serves as a cover for the fluid cavity and is composed of material which automatically reseals itself after having been penetrated by a needle.

The needle-impenetrable housing also contains an opening for connection with a conduit, such as a catheter, which permits fluid communication between the fluid cavity and the conduit. The proximal end of the conduit may be slidably mounted on an outlet stem protruding from the needle-impenetrable housing. The distal end of the catheter is positioned at the selected body site, such as in a large vein.

The port is surgically inserted under the skin with the self-sealing septum portion positioned outwardly towards the skin of the patient. The self-sealing septum, otherwise known as an access point, allows for repeated, non-destructive access to the fluid cavity by a needle.

Once the port and catheter have been implanted beneath the skin of a patient, fluids may be infused into the fluid cavity by means of a needle that is passed in order: i) through the skin of the patient; ii) through the self-sealing septum portion; and iii) into the fluid cavity of the port. The fluid injected into the fluid cavity travels through the outlet of the housing and into the proximal end of the catheter mounted thereon, shortly thereafter arriving at the selected body site where the distal end of the catheter is located.

Fluids may also be withdrawn from the body of a patient through the subject medical port. This is accomplished by piercing the skin of the patient and the self-sealing septum portion of the port with a needle, or any other needle used to deliver and withdraw fluids, such as a Huber needle, and applying negative pressure. This causes the fluid, such as blood, to be drawn through the catheter into the fluid cavity and out of the body of the patient through the needle.

Although such ports allow healthcare providers easy access to a patient's blood stream repeatedly and safely, the orientation of the port can change underneath the skin due to loosened sutures or other causes, and because the fluid cavity can only be accessed from the septum, or multiple septa, on a single side of the port(s), a port that has become inverted or is otherwise is in an upside down position, is rendered useless. Also, if the soft tissue is too loose, it is generally difficult to ensure that the septum on a single side of the port is facing outwardly towards the skin where it can be accessed by a needle. Further, for ports having a septum on a single side of the housing, or in the case of multiple connected ports with multiple septa on a single side of the housings, it is difficult to locate the septum portion with the tip of the needle when the port is covered by the skin of the patient.

Unfortunately, in order to reposition the port so that the single septum portion is facing in the proper direction outwardly towards the skin, surgery must be performed. In some instances, the port needs to be replaced altogether. Such procedures typically require general anesthesia, which expose the patient to serious risks such as death, heart attacks, strokes, blood clots, pneumonia and others. These intrusive procedures also create scars that may be painful or unsightly, and pose a risk of infection. In addition, when the surgeon is typically placing a conventional port having a septum on a single side of the housing, the surgeon must perform additional steps in an attempt to anchor the port to the surrounding tissue to decrease the risk of movement. These additional steps are delicate and increase the complexity, difficulty and duration of the procedure and hence the amount of time the patient is under sedation, thereby increasing risks to the patient. Accordingly, there is a need for an improved implantable medical port which overcomes the above-noted problems. The present disclosure fulfills these needs and provides other related advantages.

### BRIEF DESCRIPTION OF THE DRAWINGS

Conventional implantable medical ports are depicted in FIGS. 1-5. The disclosed implantable medical port is readily understood when read in conjunction with FIGS. 6-18. It should be noted that the implantable medical port is not limited to any of the illustrative embodiments shown in the drawing figures, but rather should be construed in breadth and scope in accordance with the disclosure provided herein.

FIG. 1 is a perspective view of a conventional implantable medical port.

FIG. 2 is a cross-sectional view of a conventional implantable medical port.

FIG. 3 is a side cross-sectional view of a conventional medical port that is subcutaneously implanted. The medical port has been positioned so that the septum portion on a single side of the housing is facing outwardly towards the skin line where it can be accessed by a needle.

FIG. 4 is side cross-sectional view of the implantable medical port of FIG. 3 that has become inverted so that the septum portion on a single side of the housing is facing inwardly away from the skin line, necessitating a surgical procedure to reposition the medical port so that the septum portion faces outwardly towards the skin.

FIG. 5 is a schematic, side cross-sectional view of a conventional medical port subcutaneously implanted. A needle has been passed in order: i) through the skin; ii) through the single septum portion; and iii) into the fluid cavity, allowing fluid to be injected into the cavity, and expelled through the catheter into to a blood vessel.

FIG. 6 is a cross-sectional view of an illustrative embodiment of the implantable medical port having first and second septum portions on opposing sides of the housing.

FIG. 7 is a side cross-sectional view of the implantable medical port of FIG. 6 subcutaneously implanted and positioned so that the first septum portion is facing outwardly towards the skin line and the second septum portion is facing inwardly away from the skin line.

FIG. 8 is a side cross-sectional view of the implantable medical port of FIG. 7 that has become inverted, however, the second septum portion is now facing outwardly towards the skin line, providing an access point for a needle to enter the fluid cavity.

FIG. 9 is a cross-sectional view of an illustrative embodiment of the implantable medical port having first and second opposing self-sealing septum portions and a substantially needle-impenetrable member floating inside the fluid cavity to prevent the needle from inadvertently puncturing a septum portion other than the initial septum that is punctured to gain access to the fluid cavity.

FIG. 10 is a cross-sectional view of an illustrative embodiment of the implantable medical port having first and second opposing self-sealing septum portions and a substantially needle-impenetrable member that is slidably engaged with rail members positioned on the side walls of the fluid cavity to prevent the needle from inadvertently puncturing a septum portion other than the initial septum that is punctured to gain access to the fluid cavity.

FIG. 11 is a cross-sectional view of an illustrative embodiment of the implantable medical port having first and second opposing self-sealing septum portions and a hinged substantially needle-impenetrable member that is pivotable between a raised and lowered position in the fluid cavity to prevent the needle from inadvertently puncturing a septum portion other than the initial septum that is punctured to gain access to the fluid cavity.

FIG. 12 is a cross-sectional view of an embodiment of the implantable medical port having first and second opposing self-sealing septum portions and a substantially needle-impenetrable member that is fixed to at least a portion of the fluid cavity wall to prevent the needle from inadvertently puncturing a septum portion other than the initial septum that is punctured to gain access to the fluid cavity.

FIG. 13 is a cross-sectional view of an embodiment of the implantable medical port having a self-containing, needle-penetrable, self-sealing pouch defining a fluid cavity.

FIG. 14 is a side cross-sectional view of the implantable medical port of FIG. 13 subcutaneously implanted.

FIG. 15 is a side cross-sectional view of the implantable medical port of FIG. 13 wherein the fluid cavity contains a substantially needle-impenetrable member.

FIG. 16 is a side cross-sectional view of the implantable medical port of FIG. 15 subcutaneously implanted.

FIG. 17 is a side cross-sectional view of the implantable medical port of FIG. 15, wherein the substantially needle-impenetrable member is unattached to the fluid cavity walls.

FIG. 18 is a side cross-sectional view of the implantable medical port of FIG. 15 wherein the substantially needle-impenetrable member is attached to at least a portion of the fluid cavity walls.

Disclosed is an implantable medical port and methods of using the medical port to deliver an agent to, or withdrawn an agent from, the body of a subject. The desired agent delivered to and/or withdrawn from the subject may comprise medicines, treatments, or any other fluid.

The implantable medical port may include a housing enclosing or otherwise defining at least one fluid cavity. In certain embodiments, at least a portion of the housing comprises a substantially needle-impenetrable material. The phrase “substantially needle-impenetrable” means that the member will not be penetrated, pierced or cracked by a needle inserted into the fluid cavity with typical force by, for example, a doctor, nurse or other person. The housing may also comprise needle-penetrable, self-sealing septum portions, otherwise referred to as needle “receiving ports” or “access points”.

In certain embodiments, the housing comprises first and second opposing septum portions connected by the substantially needle-impenetrable portions of the housing. The needle-impenetrable material may comprise a biocompatible material such as metals, metal alloys, polymers, ceramics, composite, or any other suitably biocompatible material known in the art.

The housing may have side wall portions comprising needle-impenetrable material and top and bottom portions comprising needle-penetrable septum portions. The needle-impenetrable and needle-penetrable portions of the housing define the fluid cavity. Regardless of whether the implantable medical port is right-side-up or upside-down, the at least one fluid cavity can be accessed by a needle.

In certain embodiments, the housing comprises three or more needle-penetrable septum portions. In further embodiments, the housing comprises four or more needle-penetrable septum portions. In yet further embodiments, the housing comprises five or more needle-penetrable septum portions. The housing may comprise an unlimited number of septum portions on any or all sides to access at least one fluid cavity. In some embodiments, the septum portions are separate and distinct portions. In further embodiments, the septum portions are distinct portions separated by a substantially needle impenetrable material. The housing may have a square or rectangle shape with septum portions on at least two sides of the housing. The increased number of septum portions on the housing provide an increased needle penetration area, thereby mitigating or eliminating the need to surgically reposition a port that has become inverted or otherwise changed its orientation under the skin such that the septum portion cannot be accessed by a needle.

The housing and the at least one fluid cavity contained therein may comprise any suitable shape or size. By way of illustration, but not limitation, the housing may have a substantially cylindrical shape having septum portions on opposing ends and longitudinal walls extending therebetween made of a substantially needle-impenetrable material. In certain embodiments, the longitudinal walls may also comprise at least one needle-penetrable septum portion. In further embodiments, the housing may have a substantially square or rectangle shape having needle-impenetrable and needle-penetrable portions on at least two sides. In further embodiments, the housing may have a substantially circular or oval shape having a plurality of needle-impenetrable and needle-penetrable portions.

In certain embodiments, the housing comprises a self-containing needle-penetrable septum enclosing at least one fluid cavity. According to this embodiment, the housing does not include a needle-impenetrable portion. Thus, the fluid cavity can be accessed by a needle regardless of the position or orientation of the port under the skin of a patient. The housing according to this embodiment may take any shape or size. In certain embodiments, the housing is substantially oval-shaped for patient comfort.

In certain embodiments, at least a portion of the septum is flexible and made of a material which is penetrable by a hypodermic needle or any other needle, such as a Huber needle, for delivering and/or withdrawing fluid from the cavity. At least a portion of the septum is substantially self-sealing upon removal of the needle therefrom. The septum portion may be manufactured from many different material such as thermoplastics, elastomers, polyurethanes, silicones, vinyl, rubbers, or combinations thereof.

The housing may contain an opening for connection with a conduit, such as a catheter. The conduit has proximal and distal ends and a longitudinal axis extending therebetween. The proximal end of the conduit is connected to an opening on the housing. By way of illustration, but not limitation, the opening may include an outlet stem protruding from the housing. The proximal end of the conduit may be slidably mounted onto the outlet stem of the housing and the distal end of the conduit is positioned at the selected body site, such as in a large vein. The conduit permits fluid communication between the fluid cavity of the port and the selected body site.

Once the medical port and catheter have been implanted beneath the skin of a patient, fluids may be infused into the fluid cavity by means of a needle which is passed in order: i) through the skin of the patient; ii) through one of the septum portions; and iii) into the fluid cavity. The infused fluid is then directed through the opening of the housing to which the proximal end of the catheter is attached or otherwise connected, travels through the longitudinal axis of the catheter lumen until reaching the distal end of the catheter that is positioned at the desired location. Also, fluids may be withdrawn from the body of a patient by piercing the skin of the patient and the septum with a hypodermic needle and applying negative pressure. This causes the fluid, such as blood, to be drawn through the catheter into the fluid cavity and out of the body of the patient through the needle.

In certain embodiments, the housing comprises a single cavity and a single opening that is connected to at least one conduit. The single cavity can be accessed by a needle from two or more septum portions of the housing. In other embodiments, the housing comprises two or more fluid cavities and one or more openings connected to one or more conduits.

The at least one fluid cavity may contain at least one substantially needle-impenetrable member to prevent a needle from puncturing septum portions other than the septum portion initially punctured by the needle to gain access to the fluid cavity. The phrase “substantially needle-impenetrable” means that the member will not be penetrated, pierced or cracked by a needle inserted into the fluid cavity with typical force by, for example, a doctor, nurse or other person. The substantially needle-impenetrable member may comprise a solid, semi-solid or hollow sheet or mesh material with any desired shape or size. In certain embodiments, the at least one substantially needle-impenetrable member is X-shaped or crisscross-shaped. The at least one substantially needle-impenetrable member may be fixed or movable within the fluid cavity. The substantially needle-impenetrable member may be attached or unattached to the fluid cavity wall. The at least one substantially needle-impenetrable member may be flexible or rigid. The substantially needle-impenetrable member may also be made of multiple pieces of the same or combination of materials which fit together to form the needle-impenetrable member. The at least one substantially needle-impenetrable member may be substantially coextensive with, or otherwise configured to fit the dimensions of, the walls of the fluid cavity.

The at least one substantially needle-impenetrable member may be in a fixed or unfixed position inside the at least one fluid cavity. In certain embodiments, at least a portion of the at least one substantially needle-impenetrable member is affixed to at least a portion of the walls of the fluid cavity in any suitable manner known in the art. This affixment can be made over any pre-selected portion of the at least one substantially needle-impenetrable member.

When the housing of the implantable medical port comprises first and second opposing septum portions, the at least one substantially needle-impenetrable member may be positioned in the fluid cavity so that when the needle punctures the first septum portion to gain access to the fluid cavity it does not puncture the opposing second septum portion of the housing.

In certain embodiments, the at least one substantially needle-impenetrable member is discontinuously attached to the walls of the fluid cavity. In other words, not all portions of the at least one substantially needle-impenetrable member are in contact with the walls of the fluid cavity. This discontinuous attachment provides channels in the at least one substantially needle-impenetrable member through which fluid may flow freely from either side of the needle-impenetrable member in the fluid cavity.

In certain embodiments, the at least one substantially needle-impenetrable member includes a plurality of perforations, slits, passages or openings that allow the fluid in the cavity to flow through the needle-impenetrable member so that fluid may flow freely from either side of the needle-impenetrable member in the fluid cavity.

In certain embodiments, at least one end of the at least one substantially needle-impenetrable member is hinged so as to be pivotable between a raised and lowered position in the fluid cavity.

In other embodiments, the at least one substantially needle-impenetrable member is guided by at least one guide member inside the at least one fluid cavity. In further embodiments, the at least one substantially needle-impenetrable member is guided by at least one guide member that passes through the at least one substantially needle-impenetrable member. In yet further embodiments, the at least one substantially needle-impenetrable member rotates about a central axis or shaft.

In certain embodiments, the at least one substantially needle-impenetrable member is in a substantially unfixed position inside the at least one fluid cavity. In further embodiments, the at least one substantially needle-impenetrable member is freely floating inside the fluid cavity. The at least one substantially needle-impenetrable member may be constructed of a material that permits it to float when the cavity contains fluid. According to this embodiment, the at least one substantially needle-impenetrable member should have a substantially similar specific gravity as the injected fluid to allow buoyancy behavior by the at least one substantially needle-impenetrable member.

Floating within the fluid cavity permits the at least one substantially needle-impenetrable member to be moved or easily repositioned by the needle accessing the cavity so as to increase the space in the vicinity of the needle tip. This increase in space facilitates the addition or removal of fluid from the cavity.

The at least one substantially needle-impenetrable member may be composed of a variety of materials including metals, metal alloys, polymers, ceramics, composite, or any other suitable material known in the art. According to certain embodiments, the at least one substantially needle-impenetrable member is manufactured from polymeric materials.

In some embodiments, at least one substantially fluid-tight septum is positioned within at least one portion of the lumen of the catheter to prevent or limit flow of a fluid through the catheter. When a syringe or other device is used to create an increase in pressure inside the lumen of the catheter, the substantially fluid-tight septum at least partially opens and allows fluid to flow into or from the catheter. The phrase “substantially fluid-tight” means that the septum prevents or mitigates leaking or passage of liquid through the septum, the septum may not close sufficiently to prevent passage of gases. The at least one substantially fluid-tight septum may include a slit that opens under pressure from the syringe and permits two-way fluid communication therethrough.

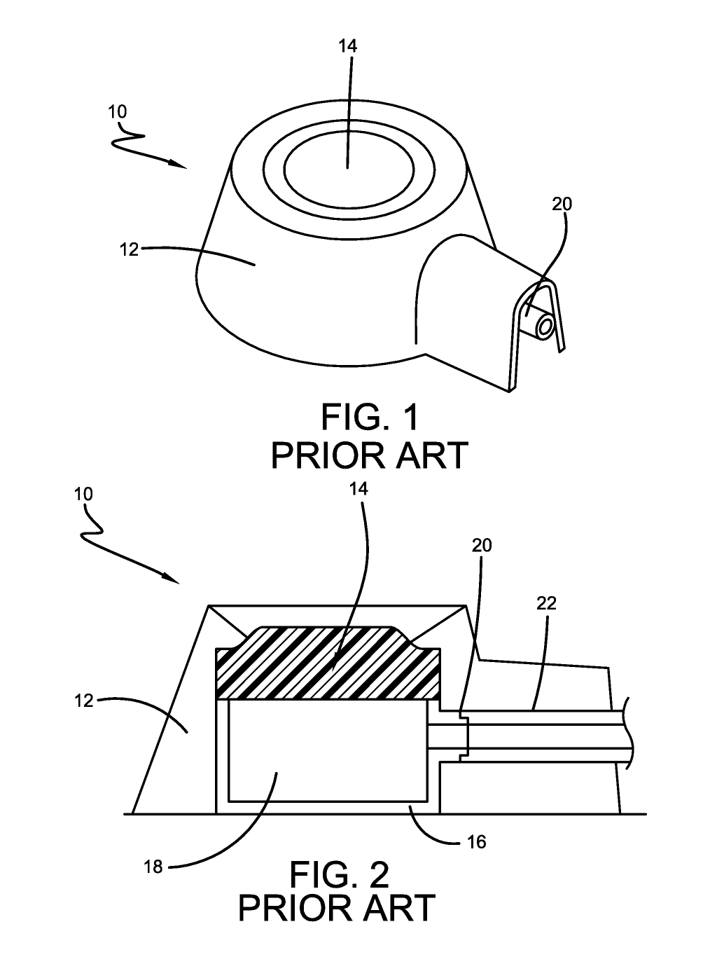

Conventional implantable medical ports are described in FIGS. 1-5.

FIG. 1 is a perspective view of a conventional implantable medical port 10. Implantable medical port 10 includes needle-impenetrable outer housing 12 and needle-penetrable septum portion 14 on a single side of housing 12. Septum portion 14 partially encloses a fluid cavity (not shown) and is composed of material which reseals itself upon removal of a needle therefrom. Needle-impenetrable outer housing 12 includes outlet 20 for connection with conduit (not shown).

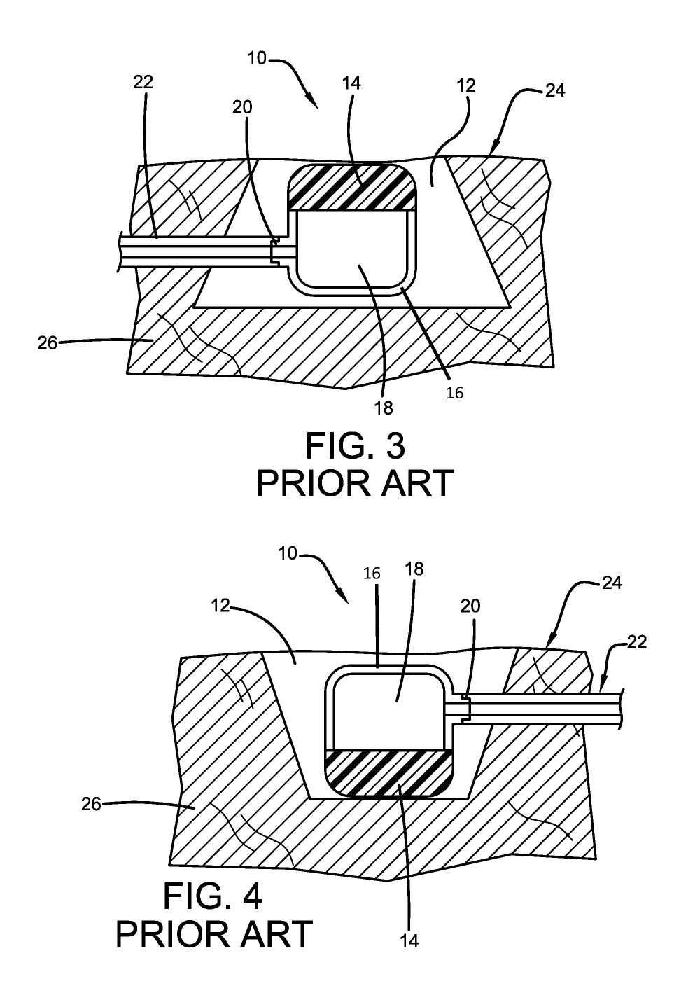

FIG. 2 is a cross-sectional view of a conventional implantable medical port 10. Implantable medical port 10 includes outer housing 12 containing inner housing 16 defined by needle-impenetrable portion and a single needle-penetrable septum portion 14. Single septum portion 14 partially encloses fluid cavity 18 and is composed of material which reseals itself upon removal of a needle therefrom. Inner housing 16 includes outlet 20 for connection with conduit 22.

FIG. 3 is a side cross-sectional view of a conventional implantable medical port 10 that is subcutaneously implanted. Implantable medical port 10 has single needle-penetrable septum portion 14 facing outwardly towards skin line 24. Sutures may be used to affix implantable medical port 10 to surrounding tissue 26. After port 10 is implanted under skin line 24, upper surface of single septum portion 14 is beneath skin line 24. Single septum portion 14 may be repeatedly punctured for creating a percutaneous passageway from the exterior of the skin of a patient into fluid cavity 18. Outlet 20 provides a fluid-communicative passageway from fluid cavity 18 to outlet 20 and conduit 22 coupled thereto.

FIG. 4 is side cross-sectional view of implantable medical port 10 of FIG. 3 that has become inverted under skin line 24 so that single needle-penetrable septum portion 14 is facing inwardly away from skin line 24 and needle-impenetrable portion of inner housing 16 is facing outwardly towards skin line 24. A surgical procedure is necessary to reposition implantable medical port 10 so that single septum portion 14 is facing outwardly towards skin line 24.

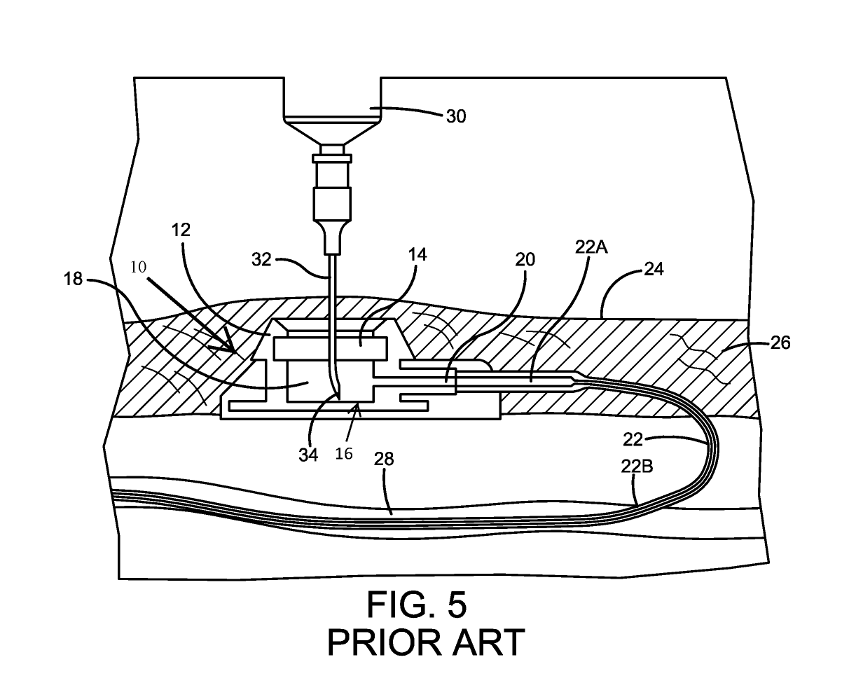

FIG. 5 is a side cross-sectional view of a conventional medical port 10 that is subcutaneously implanted. Implantable medical port 10 has single needle-penetrable septum portion 14 facing outwardly towards skin line 24. Sutures may be used to affix implantable medical port 10 to surrounding tissue 26. After port 10 is implanted under skin line 24, upper surface of single septum portion 14 is beneath skin line 24. Single septum portion 14 may be repeatedly punctured for creating a percutaneous passageway from the exterior of the skin of the patient into fluid cavity 18. Outlet 20 provides a fluid-communicative passageway from cavity 18 to outlet 20 and catheter 22 coupled thereto. Outlet 20 is in connection with proximal end 22A of catheter 22. Distal end 22B of catheter 22 is positioned in blood vessel 28. Syringe 30 fitted with a needle 32 penetrates skin line 24, single septum portion 14, and enters fluid cavity 18. Needle tip 34 is stopped by needle-impenetrable portion 16 of housing. Fluid is injected into cavity 18 by needle 32 and expelled through catheter 22 into to blood vessel 28.

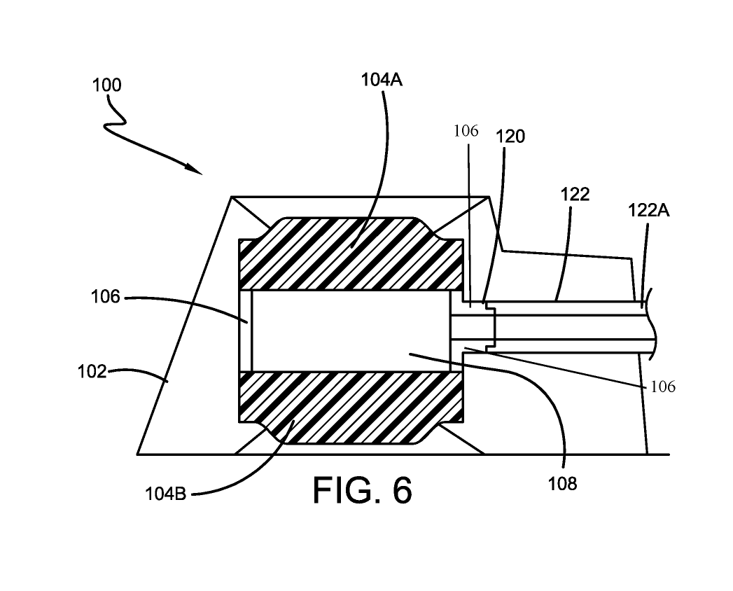

The presently disclosed implantable medical port is readily understood when read in conjunction with illustrative FIGS. 6-18. It should be noted that the implantable medical port is not limited to any of the embodiments shown in the drawing figures, but rather should be construed in breadth and scope in accordance with the disclosure provided herein.

FIG. 6 is a cross-sectional view of an illustrative embodiment of the implantable medical port 100. Implantable medical port 100 includes outer housing 102 containing an inner housing 106 defined by substantially needle-impenetrable portions and needle-penetrable first 104A and second 104B opposing septum portions. First 104A and second 104B septum portions partially enclose fluid cavity 108 and are composed of material which reseals itself upon removal of a needle therefrom. Inner housing includes opening 120 for connection with proximal end 122A of conduit 122.

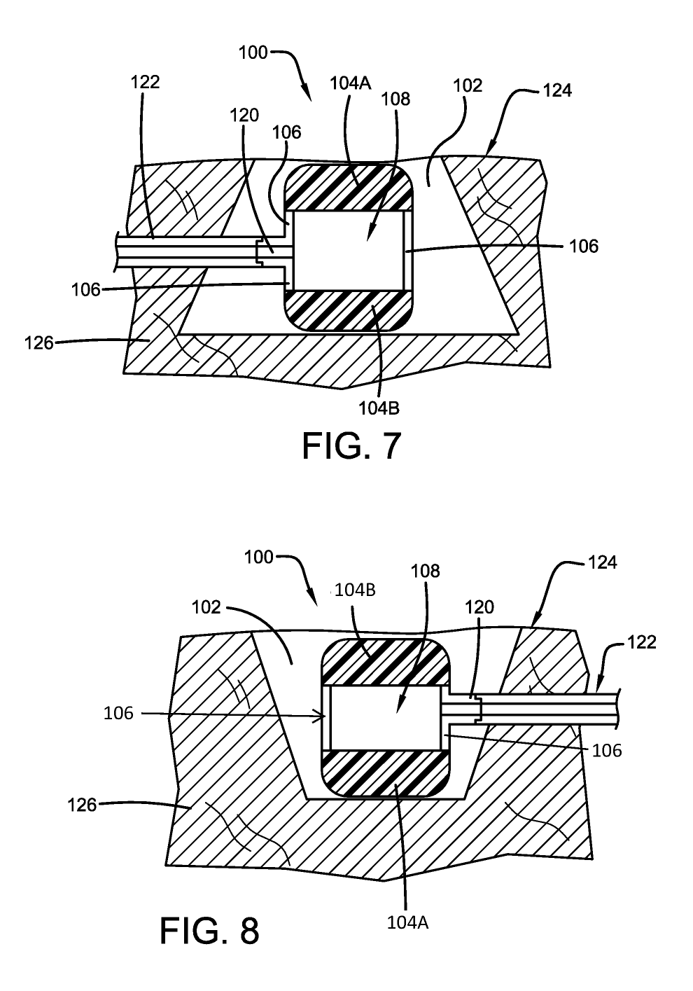

FIG. 7 is a cross-sectional view of an illustrative embodiment of the implantable medical port 100 that is subcutaneously implanted. Implantable medical port 100 includes outer housing 102 containing an inner housing 106 defined by substantially needle-impenetrable portions and needle-penetrable first 104A and second 104B opposing septum portions. First 104A and second 104B septum portions partially enclose fluid cavity 108 and are composed of material which reseals itself upon removal of a needle therefrom. Sutures may be used to affix implantable medical port 100 to surrounding tissue 126. After port 100 is implanted under skin line 124, surface of first 104A septum portion is facing outwardly towards skin line 124 and surface of second 104B septum portion is facing inwardly away from skin line 124. First 104A septum portion may be repeatedly punctured for creating a percutaneous passageway from the exterior of the skin of the patient into fluid cavity 108. Outlet 120 provides a fluid-communicative passageway from cavity 108 to outlet 120 and conduit 122 coupled thereto. Regardless of whether the implantable medical port is right-side-up or upside-down, fluid cavity 108 can be accessed by a needle via first 104A or second 104B septum portions.

FIG. 8 is side cross-sectional view of implantable medical port 100 of FIG. 7 that has become inverted under skin line 124 so that second 104B septum portion is facing outwardly towards skin line 124 and first 104A septum portion is facing inwardly away from skin line 124. Regardless of whether the implantable medical port is right-side-up or upside-down, fluid cavity 108 can be accessed by a needle via first 104A or second 104B septum portions.

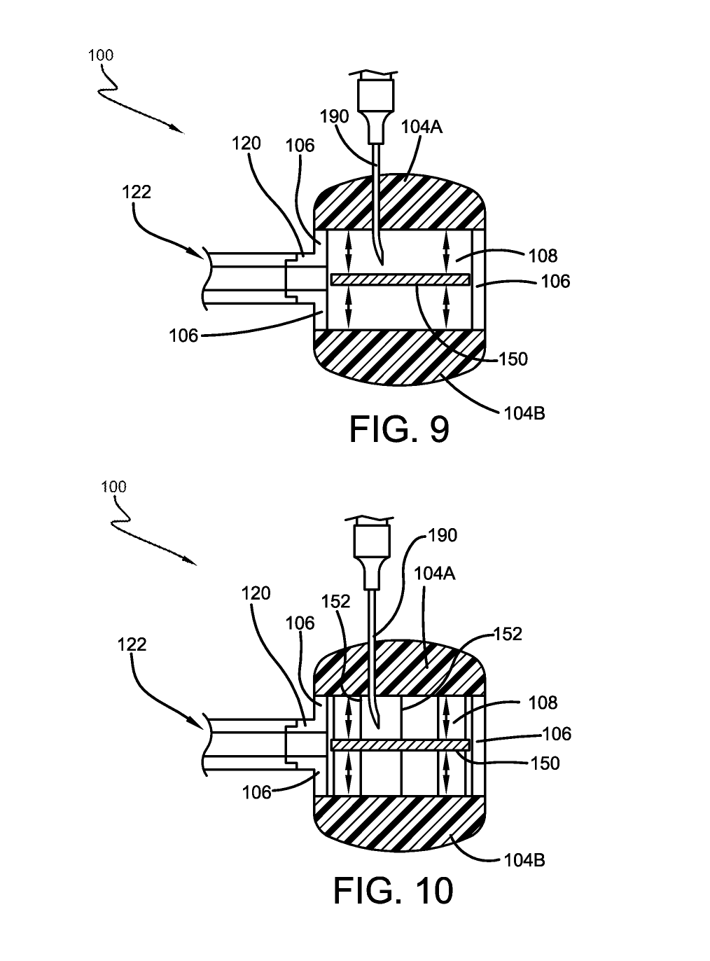

FIG. 9 is a cross-sectional view of an illustrative embodiment of the implantable medical port 100. Implantable medical port 100 includes housing defined by needle-impenetrable portions 106 and needle-penetrable opposing first 104A and second 104B septum portions. First 104A and second 104B septum portions partially enclose fluid cavity 108 and are composed of material which reseals itself upon removal of needle 190 therefrom. Fluid cavity 108 contains a substantially needle-impenetrable member 150 which is freely moving between the first 104A and second septum 104B portions and prevents needle 190 from puncturing second 104B septum portion.

FIG. 10 is a cross-sectional view of an illustrative embodiment of the implantable medical port 100. Implantable medical port 100 includes housing defined by needle-impenetrable portions 106 and needle-penetrable first 104A and second 104B septum portions. First 104A and second 104B septum portions partially enclose fluid cavity 108 and are composed of material which reseals itself upon removal of needle 190 therefrom. Fluid cavity 108 contains substantially needle-impenetrable member 150 slidably engaged with a plurality of guide members 152 such as frame, rail or rod members, inside fluid cavity 108 to prevent needle 190 from puncturing second 104B septum portion.

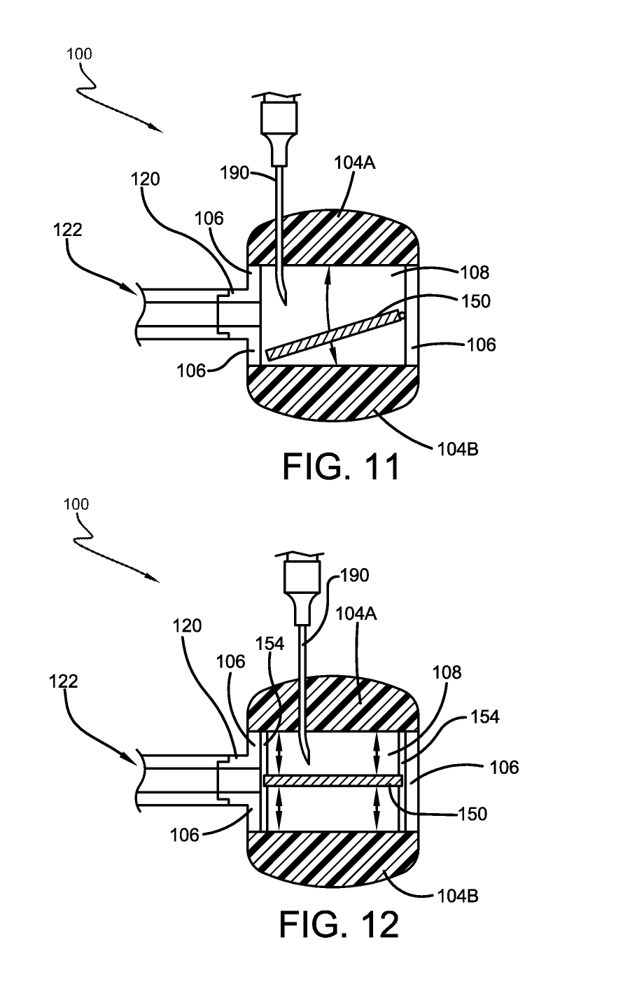

FIG. 11 is a cross-sectional view of an illustrative embodiment of the implantable medical port 100. Implantable medical port 100 includes housing defined by needle-impenetrable portions 106 and needle-penetrable first 104A and second 104B septum portions. First 104A and second 104B septum portions partially enclose fluid cavity 108 and are composed of material which reseals itself upon removal of needle 190 therefrom. Fluid cavity 108 contains substantially needle-impenetrable member 150 that is pivotable between a raised and lowered position in fluid cavity 108 to prevent needle 190 from puncturing second 104B septum portion.

FIG. 12 is a cross-sectional view of an illustrative embodiment of the implantable medical port 100. Implantable medical port 100 includes housing defined by needle-impenetrable portions 106 and needle-penetrable opposing first septum portion 104A and second septum portion 104B. Septum portions 104A and 104B partially enclose fluid cavity 108 and are composed of material which reseals itself upon removal of needle 190 therefrom. Fluid cavity 108 contains needle-impenetrable member 150. At least a portion of the outer perimeter of needle-impenetrable member 150 is affixed to at least a portion of the wall of fluid cavity 108. This affixment can be made over any pre-selected portion of needle-impenetrable member 150. Needle-impenetrable member 150 prevents needle 190 from puncturing second septum portion 104B.

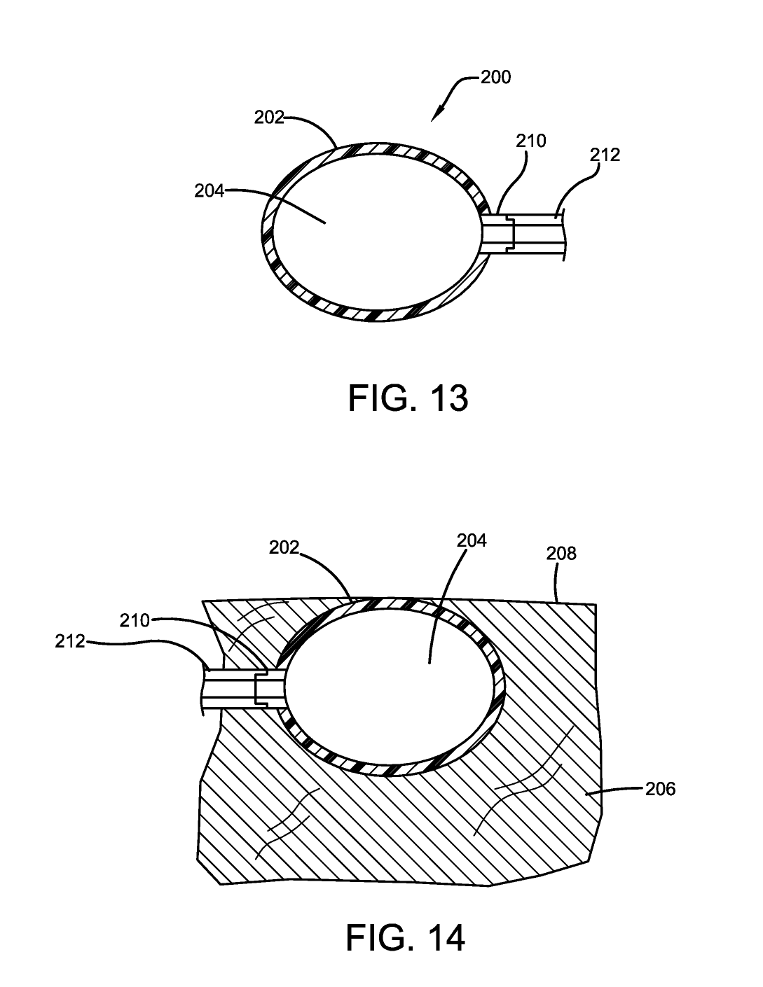

FIG. 13 is a cross-sectional view of an illustrative embodiment of the implantable medical port 200 having a self-containing needle-penetrable housing 202 enclosing fluid cavity 204. Fluid cavity 204 can be accessed by a needle regardless of how implantable medical port 200 is oriented under the skin of a patient. Outlet 210 provides a fluid-communicative passageway from cavity 204 to outlet 210 and conduit 212 coupled thereto.

FIG. 14 is a cross-sectional view of implantable medical port 200 that is subcutaneously implanted. Implantable medical port 200 includes self-containing needle-penetrable housing 202 enclosing fluid cavity 204. Fluid cavity 204 can be accessed by a needle regardless of how the implantable medical port 200 is oriented under skin line 208. Self-containing needle-penetrable housing 202 may be repeatedly punctured for creating a percutaneous passageway from the exterior of the skin of the patient into fluid cavity 204. Outlet 210 provides a fluid-communicative passageway from cavity 204 to outlet 210 and conduit 212 coupled thereto.

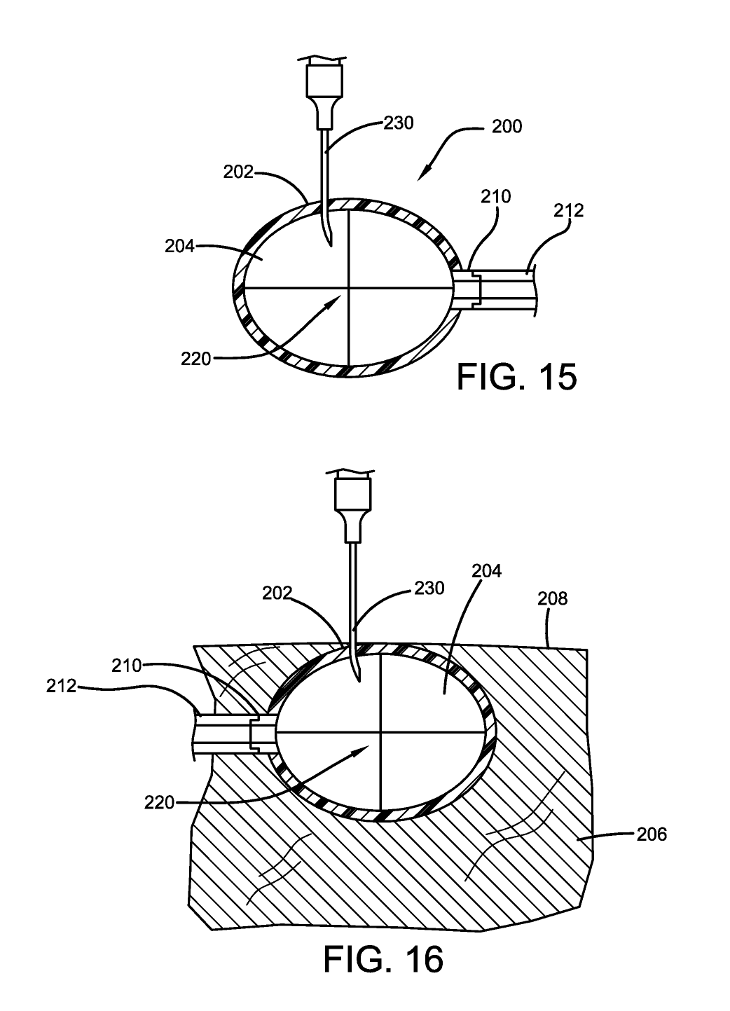

FIG. 15 is a cross-sectional view of implantable medical port 200. Implantable medical port 200 includes self-containing needle-penetrable housing 202 enclosing fluid cavity 204. Self-containing needle-penetrable housing 202 may be repeatedly punctured for creating a percutaneous passageway from the exterior of the skin of the patient into fluid cavity 204. Outlet 210 provides a fluid-communicative passageway from fluid cavity 204 to outlet 210 and conduit 212 coupled thereto. Fluid cavity 204 contains needle-impenetrable member 220 fixed to at least a portion of the housing 202. Needle-impenetrable member 220 prevents needle 230 from puncturing a portion of self-containing housing 202 other than the portion initially punctured to gain access to fluid cavity 204.

FIG. 16 is a cross-sectional view of implantable medical port 200 that is subcutaneously implanted. Implantable medical port 200 includes self-containing needle-penetrable housing 202 enclosing fluid cavity 204. Fluid cavity 204 can be accessed by a needle regardless of how the implantable medical port 200 is positioned under skin line 208. Self-containing needle-penetrable housing 202 may be repeatedly punctured for creating a percutaneous passageway from the exterior of the skin of the patient into fluid cavity 204. Outlet 210 provides a fluid-communicative passageway from cavity 204 to outlet 210 and conduit 212 coupled thereto. Fluid cavity 204 contains needle-impenetrable member 220 fixed to at least a portion of the walls of housing 202. Needle-impenetrable member 220 prevents needle 230 from puncturing a portion of self-containing housing 202 other than the portion initially punctured to gain access to fluid cavity 204.

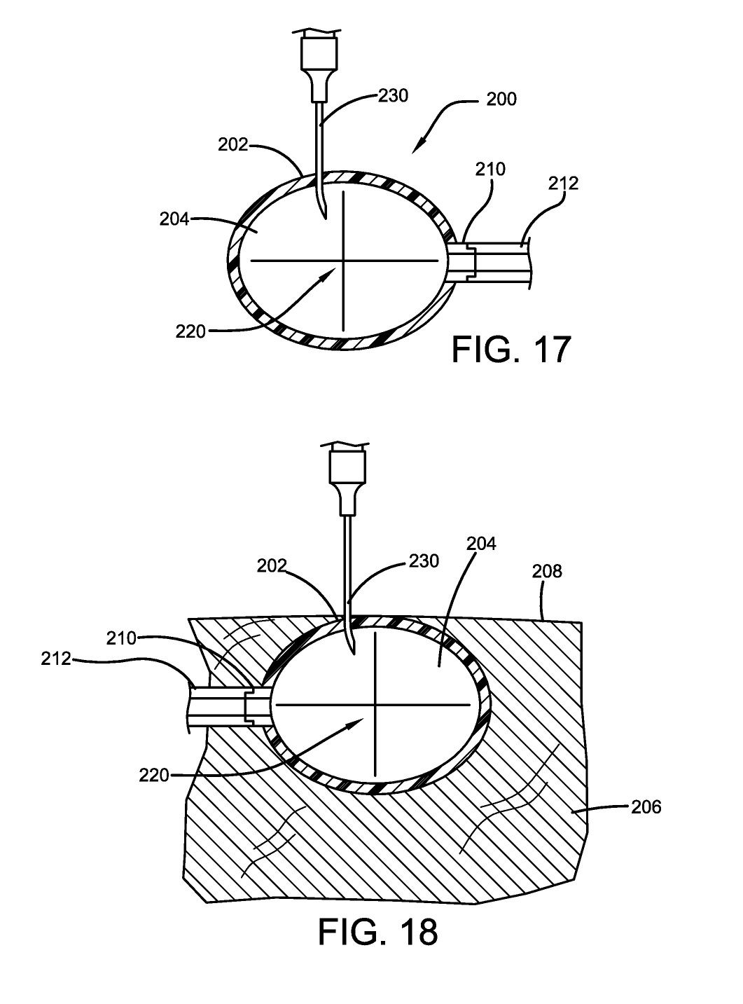

FIG. 17 is a cross-sectional view of implantable medical port 200. Implantable medical port 200 includes self-containing needle-penetrable housing 202 enclosing fluid cavity 204. Self-containing needle-penetrable housing 202 may be repeatedly punctured for creating a percutaneous passageway from the exterior of the skin of the patient into fluid cavity 204. Outlet 210 provides a fluid-communicative passageway from cavity 204 through outlet 210 and into conduit 212 coupled thereto. Fluid cavity 204 contains a substantially needle-impenetrable member 220 that is not affixed to housing 202. Substantially needle-impenetrable member 220 prevents needle 230 from puncturing a portion of self-containing, needle-penetrable housing 202 other than the portion initially punctured to gain access to fluid cavity 204.

FIG. 18 is a cross-sectional view of implantable medical port 200 that is subcutaneously implanted. Implantable medical port 200 includes self-containing, needle-penetrable housing 202 enclosing fluid cavity 204. Fluid cavity 204 can be accessed by a needle regardless of how the implantable medical port 200 is positioned under skin line 208. Self-containing needle-penetrable housing 202 may be repeatedly punctured for creating a percutaneous passageway from the exterior of the skin of the patient into fluid cavity 204. Outlet 210 provides a fluid-communicative passageway from cavity 204 to outlet 210 and conduit 212 coupled thereto. Fluid cavity 204 contains a substantially needle-impenetrable member 220 that is not affixed to housing 202. Substantially needle-impenetrable member 220 prevents needle 230 from puncturing a portion of self-containing, needle-penetrable housing 202 other than the portion initially punctured to gain access to fluid cavity 204.

While the implantable medical port, and methods of using the implantable medical port have been described in connection with various embodiments, it is to be understood that other similar embodiments may be used or modifications and additions may be made to the described embodiments for performing the same function. Furthermore, the various illustrative embodiments may be combined to produce the desired results. Therefore, the implantable medical port, and methods of using the implantable medical port should not be limited to any single embodiment.

It will be understood that the embodiments described herein are merely exemplary, and that one skilled in the art may make variations and modifications without departing from the spirit and scope of the invention. All such variations and modifications are intended to be included within the scope of the invention as described hereinabove. Further, all embodiments disclosed are not necessarily in the alternative, as various embodiments of the invention may be combined to provide the desired result.

1. An implantable medical port comprising:
a housing defining at least one fluid cavity;
wherein the housing comprises two or more needle-penetrable, self-sealing septum portions that provide selective access to the at least one fluid cavity;
wherein a single fluid cavity is configured to be accessed by a needle from two or more septum portions of the housing; and
at least one outlet for connection with a conduit.

2. The implantable medical port of claim 1, wherein the housing comprises three or more needle-penetrable septum portions to provide selective access to the at least one fluid cavity.

3. The implantable medical port of claim 1, wherein the housing comprises four or more needle-penetrable septum portions to provide selective access to the at least one fluid cavity.

4. The implantable medical port of claim 1, wherein the housing comprises five or more needle-penetrable septum portions to provide selective access to the at least one fluid cavity.

5. The implantable medical port of claim 1, wherein the two or more needle-penetrable, self-sealing septum portions comprise silicone.

6. The implantable medical port of claim 1, wherein the housing comprises two septum portions on opposing sides of the housing and a longitudinal axis extending therebetween comprising the at least one substantially needle-impenetrable portion.

7. The implantable medical port of claim 6, wherein the at least one fluid cavity contains at least one substantially needle-impenetrable member located therein.

8. The implantable medical port of claim 7, wherein the at least one substantially needle-impenetrable member comprises a solid sheet or mesh.

9. The implantable medical port of claim 7, wherein the at least one substantially needle-impenetrable member is freely floating within the fluid cavity.

10. The implantable medical port of claim 7, wherein the at least one substantially needle-impenetrable member is in a fixed position.

11. The implantable medical port of claim 7, wherein the at least one substantially needle-impenetrable member is affixed to at least a portion of the at least one fluid cavity.

12. The implantable medical port of claim 11, wherein the at least one substantially needle-impenetrable member is discontinuously affixed to the at least one fluid cavity.

13. The implantable medical port of claim 7, wherein the at least one substantially needle-impenetrable member includes a plurality of perforations to allow fluid to flow from either side of the substantially needle-impenetrable member.

14. The implantable medical port of claim 7, wherein, one end of the at least one substantially needle-impenetrable member is hingedly connected to the housing so as to be pivotable between a raised and lowered position in the at least one fluid cavity.

15. The implantable medical port of claim 7, wherein the at least one substantially needle-impenetrable member is guided by at least one guide member inside the at least one fluid cavity.

16. The implantable medical port of claim 15, wherein the at least one guide member inside comprises at least one of: i) a frame; ii) a rail; or iii) a rod.

17. The implantable medical port of claim 7, wherein the at least one substantially needle-impenetrable member is not fixedly attached to walls of the at least one fluid cavity.

18. An implantable medical port comprising:
a housing defining at least one fluid cavity;
wherein the housing comprises a self-containing needle-penetrable septum enclosing the at least one fluid cavity; and
at least one outlet for connection with a conduit.
